(19) United States
(12) Patent Application Publication
(10) Pub. No.: US 2008/0044468 A1
(43) Pub. Date: Feb. 21, 2008

(54) Processes For The Preparation Of Oral Dosage Formulations Of Modafinil

(76) Inventors: Romi Barat Singh, Uttar Pradesh (IN); Pananchukunnath Manoj Kumar, Delhi (IN); Vishnubhotla Nagaprasad, Andhra Pradesh (IN); Sunilendu Bhshan Roy, Maharashtra (IN); Rajiv Malik, Wien (AT)

Correspondence Address: RANBAXY INC., 600 COLLEGE ROAD EAST, SUITE 2100, PRINCETON, NJ, 08540, US

(21) Appl. No.: 10/522,223

(22) PCT Filed: Jul. 24, 2003
(86) PCT No.: PCT/IB03/02962

§ 371 Date: Jun. 7, 2006

(30) Foreign Application Priority Data
Jul. 25, 2002 (IN) 771/DEL/2002

(51) Int. Cl. A61K 9/48; A61K 31/165; A61K 9/14; A61P 43/00; A61K 9/20

(52) U.S. Cl. 424/463; 424/464; 424/474; 424/489; 514/618

(57) ABSTRACT

The present invention relates to a method for the preparation of bioavailable dosage form of modafinil.

### FIELD OF THE INVENTION

The technical field of the present invention relates to bioavailable dosage forms of modafinil and processes of preparation thereof.

### BACKGROUND OF THE INVENTION

Modafinil is a wakefulness-promoting agent indicated for use in narcolepsy and idiopathic hypersomnia. It also is used for improving memory and mood. Compared to amphetamines and methylphenidate, modafinil is less likely to cause jitteriness, anxiety, or excess locomotor activity. The precise mechanism of action is not fully understood but it is thought to modulate the central postsynaptic alpha₁-adrenergic receptors. However, modafinil has a different pharmacokinetic profile compared to the sympathomimetic agents, such as amphetamines and methylphenidate.

The benzhydrylsulfinyl acetamide structure of modafinil makes it insoluble in water (less than 1 mg/ml) as well as unstable at higher temperatures. These physicochemical properties decrease the drug's potential for abuse via injection or smoking, and lead to reduced cases of dependency compared to amphetamines. On the other hand, the insoluble nature of modafinil creates absorption problems, and preparation of bioavailable dosage forms of modafinil a challenging task.

Over the years, more than 40% of the potential candidates in drug discovery and research have failed to emerge as drugs due to their poor biopharmaceutic properties. Most of these are rejected due to poor solubility characteristics and further development is continued only if the new molecule has some marked advantage over the existing molecules indicated for the similar use.

The most common approach used to address the problem of insolubility is by either reducing the drug's particle size or micronizing the drug to the size of a few microns, which increases the effective exposed surface area. Dosage forms that contain micronized drug particles exhibit enhanced solubility and consequently an increase in the bioavailability of the drugs. However, technical and economical problems can arise. For example, highly micronized drug particles possess poor flow properties and an increased chance of re-agglomeration during processing. In some cases, re-agglomeration of micronized drug particles may be so problematic that the basic objective of enhancing the solubility by increasing the effective surface area may be unmet.

U.S. Pat. No. RE 37,516 discloses a method of size reduction and a pharmaceutical composition that has at least 95% of the modafinil particles having a diameter of less than 200 μm.

### SUMMARY OF THE INVENTION

In one general aspect there is provided an oral dosage form of modafinil that includes modafinil and one or more surface active agents.

Embodiments of the oral dosage from may include one or more of the following features. For example, the modafinil may include fine and coarse modafinil particles, and at least 10% of the modafinil particles are coarse modafinil particles and have diameters greater than 220 μm, and up to 90% of the modafinil particles are fine modafinil particles and have diameters less than 220 μm. At least 15% of the modafinil particles may be coarse modafinil particles and have diameters greater than 220 μm and up to 85% of modafinil particles may be fine modafinil particles and have diameters less than 220 μm. At least 25% of the modafinil particles may be coarse modafinil particles and have diameters greater than 220 μm and up to 75% of the modafinil particles may be fine modafinil particles and have diameter less than 220 μm. The total specific surface area of the fine modafinil particles may be at least 0.2 m²/g. The modafinil and the one or more surface active agents may be co-grinded and/or co-sifted.

The surface active agent may be one or more of an anionic, cationic or non-ionic surface active agent. The anionic surface active agent may be one or more of sodium lauryl sulphate, sodium laurate, dialkyl sodium sulfosuccinates, sodium stearate, potassium stearate, and sodium oleate. In particular, the anionic surface active agent may be sodium lauryl sulphate. The cationic surface active agent may be one or both of benzalkonium chloride and bis-2-hydroxyethyl oleyl amine. The non-ionic surface active agent may be one or more of polyoxyethylene sorbitan fatty acid esters, fatty alcohols, glyceryl esters, fatty acid esters of fatty alcohols, and alcohols. The fatty alcohol may be one or more of lauryl, cetyl and stearyl alcohol. The glyceryl esters may be one or more naturally occurring monoglycerides, diglycerides and triglycerides. The alcohol may be one or more of propylene glycol, polyethylene glycol, sorbitan, sucrose and cholesterol. The polyethylene sorbitan fatty acid ester may be polysorbate. The amount of surface active agent may be from about 0.2% to 10% by weight, of the total weight of the dosage form.

The oral dosage form of modafinil may further include one or more pharmaceutically inert carriers and the one or more pharmaceutically inert carriers may be one or more of cellulose derivatives, silicate derivatives, and clays. The cellulose derivative may be one or both of microcrystalline cellulose and carboxymethylcellulose. The silicate derivative may be one or more of magnesium silicate, colloidal silicon dioxide, magnesium trisilicate, and magnesium aluminum silicate. The clay may be one or more of veegum and bentonite. The amount of pharmaceutically inert carrier may be from about 2% to about 25% by weight, of total weight of the dosage form.

The oral dosage form may be a tablet, a capsule, or a pill. The oral dosage form of modafinil may further include one or more pharmaceutically inert excipients and the pharmaceutically inert excipient may be one or more of diluents, binders, disintegrants, lubricants/glidants and colors.

In another general aspect, a process for preparing an oral dosage form of modafinil includes the steps of mixing, grinding and/or sifting the mix, combining with pharmaceutically inert excipients, and compressing or filling into a suitable dosage form. The mixing includes mixing modafinil and one or both of one or more surface active agents and one or more pharmaceutically inert carriers.

Embodiments of the process may include one or more of the following features. For example, the modafinil may include fine and coarse modafinil particles, at least 10% of the modafinil particles may be coarse modafinil particles and have diameters greater than 220 μm, and up to 90% of the modafinil particles may be fine modafinil particles and have diameters less than 220 μm. At least 15% of the modafinil particles may be coarse modafinil particles and have diameters greater than 220 μm and up to 85% of the modafinil particles may be fine modafinil particles and have diameters less than 220 μm. At least 25% of the modafinil particles may be coarse modafinil particles and have diameters greater than 220 μm, and up to 75% of the modafinil particles may be fine modafinil particles and have diameters less than 220 μm. The total specific surface area of the fine modafinil particles may be at least 0.2 m²/g.

The dosage form may include one or more of a tablet, a capsule, and a pill. The tablet may be prepared by one or more of a process of wet granulation, dry granulation, or direct compression method. The dosage form may be coated with one or more functional and/or non-functional layers.

In another general aspect, a method of treating one or both of narcolepsy and idiopathic hypersomnia includes administering an oral dosage form of modafinil. The dosage form includes coarse and fine modafinil particles and one or more surface active agents. The fine modafinil particles have diameters less than 220 μm.

Embodiments of the method of treating may include one or more of the following features. For example, at least 10% of the modafinil particles may have diameters greater than 220 μm, at least 15% of the modafinil particles may have diameters greater than 220 μm, or at least 25% of the modafinil particles may have diameters greater than 220 μm. The total specific surface area of the fine modafinil particles may be at least 0.2 m²/g.

In another general aspect, a mixture includes modafinil particles and one or both of one or more surface active agents and one or more pharmaceutically inert carriers, wherein the mixture is one or both of co-grinded and co-sifted.

Embodiments of the mixture may include one or more of the following features. For example, at least 10% of the modafinil particles may be coarse and have diameters greater than 220 μm and up to 90% of the modafinil particles may be fine and have diameters less than 220 μm. At least 15% of the modafinil particles may be coarse and have diameter greater than 220 μm and up to 85% of the modafinil particles may be fine and have diameter less than 220 μm. At least 25% of the modafinil particles may be coarse and have diameters greater than 220 μm and up to 75% of the modafinil particles may be fine and have diameters less than 220 μ. The total specific surface area of the fine modafinil particles may be at least 0.2 m²/g, the fine modafinil particles having diameters less than 220 μm.

In another general aspect, an oral dosage form of modafinil includes modafinil and one or more surface active agents. The one or more surface active agents include one or more of an anionic, cationic or non-ionic surface active agent.

Embodiments of the oral dosage form may include one or more of the following features. For example, the modafinil may include fine and coarse modafinil particles, at least 10% of the modafinil particles may be coarse modafinil particles and have diameters greater than 220 μm, and up to 90% of the modafinil particles may be coarse modafinil particles and have diameters less than 220 μm. The anionic surface active agent may be one or more of sodium lauryl sulphate, sodium laurate, dialkyl sodium sulfosuccinates, sodium stearate, potassium stearate, and sodium oleate; the cationic surface active agent may be one or both of benzalkonium chloride and bis-2-hydroxyethyl oleyl amine; and the non-ionic surface active agent may be one or more of polyoxyethylene sorbitan fatty acid esters, fatty alcohols, glyceryl esters, fatty acid esters of fatty alcohols, and alcohols. The oral dosage form of modafinil may further include one or more pharmaceutically inert carriers, and the one or more pharmaceutically inert carriers may be one or more of cellulose derivatives, silicate derivatives, and clays. The oral dosage form of modafinil may further include one or more additional active pharmaceutical ingredients.

In another general aspect, an oral dosage form of modafinil includes modafinil and one or both of one or more surface active agents and one or more pharmaceutically inert carriers. The one or more surface active agents may be one or more of an anionic, cationic or non-ionic surface active agent, and the one or more pharmaceutically inert carriers may be clay.

The details of one or more embodiments of the inventions are set forth in the description below. Other features, objects, and advantages of the invention will apparent from the description and the claims.

### DETAILED DESCRIPTION OF THE INVENTION

As evident from the above description, there exists a need for simpler and cost effective approaches for preparing bioavailable dosage forms of modafinil. The inventors have now discovered that combinations of modafinil with one or more surface active agents and/or one or more pharmaceutical carriers in the preparation of pharmaceutical compositions of modafinil provide dosage forms with improved bioavailability.

In the present invention, the inventors have obtained desired dissolution profiles and bioavailability by adding either or both of one or more surface active agents and one or more pharmaceutical carriers to modafinil by one or more of co-mixing, co-sifting, and co-grinding. Modafinil used in the preparation of dosage forms is a mixture of coarse particles (diameters greater than 220 μm) and fine particles (diameters less than 220 μm) in the ratio of approximately 10:90 to 25:75 by weight. A preferred mean particle size of fines is less than 180 μm. A more preferred mean particle size of fines is approximately 15-60 μm. The ratio of coarse and fine particles may vary from a value of 10:90 to 25:75 by weight. Variations within this range do not generally affect the dissolution profile of modafinil dosage forms. The specific surface area of the fine modafinil particles should be at least 0.2 m²/gm. The combination of coarse and fine particles improves the flow properties of blend and thereby facilitates processing of dosage forms. The problems of re-agglomeration of fines and drug loss are addressed and better homogeneity is provided.

The term “surface active agent” as used herein refers to substances that improve the dissolution rate and bioavailability of modafinil by acting at the interface of the drug surface and dissolution media. For example, the term “surface active agent” can include wetting agents, solubilizers, emulsifiers, and some plasticizers. Specifically, surface active agents can include anionic, cationic, and non-ionic substances suitable as surface active agents. Suitable anionic surface active agents include those containing carboxylate, sulfonate and sulphate ions, such as sodium lauryl sulphate, sodium laurate, dialkyl sodium sulfosuccinates, particularly bis (2-ethylhexyl) sodium sulfosuccinate, sodium stearate, potassium stearate, sodium oleate and the like. Suitable cationic surfactants include those containing long chain cations, such as benzalkonium chloride, bis-2-hydroxyethyl oleyl amine and the like. Suitable non-ionic surface active agents include polyoxyethylene sorbitari fatty acid esters, fatty alcohols, such as lauryl, cetyl and stearyl alcohols, glyceryl esters, such as the naturally occurring mono-, di- and triglycerides; fatty acid esters of fatty alcohols and other alcohols, such as propylene glycol, polyethylene glycol, sorbitan, sucrose and cholesterol. Generally the surface active agent is selected from solid surface active agent so that it can be one or more of co-mixed, co-sifted, and co-grinded with modafinil. The surface-active agent may be used in amount of about 0.2% to about 10.0% by weight of the total weight of the dosage form.

The term “pharmaceutically inert carrier” refers to a substance that is physiologically acceptable and compatible with the drug and other excipients in the dosage form and has a capacity to adsorb the drug on its surface. By virtue of such adsorption, the effective surface area of the drug exposed to the dissolution media is increased manifold, which thereby increases the rate of dissolution. Such adsorption of drug on the carrier surface also prevents the re-agglomeration of drug particles due to neutralization of surface charges on the drug particles generated during milling by an inert carrier. Carriers also help in wetting the drug, which involves the uptake of water by capillary action and thereby enhances the drug dissolution further. The pharmaceutically inert carrier may be used in an amount of about 2% to about 25% by weight of the total weight of the dosage form.

Suitable pharmaceutically inert carriers include one or more of cellulose derivatives, such as microcrystalline cellulose and carboxymethylcellulose; silicate derivatives such as magnesium silicate, colloidal silicon dioxide, magnesium trisilicate, and magnesium aluminum silicate; and clays, such as veegum, bentonite; and the like.

The process of co-grinding and /or co-sifting of modafinil, and surface active agent and/or pharmaceutically inert carrier may be carried out in conventional milling instruments such as air jet mill, multi mill, ball mill, or any other method of particle attrition and/or sifting.

In one of the embodiments, the process of co-grinding modafinil and the one or more solid surface active agents and/or pharmaceutical carriers may advantageously be carried out in an accelerated air-jet mill or ball mill until the powder obtained is such that the mean particle diameter is less than or equal to 180 μm and in particular less than or equal to 60 μm.

In another embodiment, modafinil may be adsorbed onto the carrier by co-sifting the finer fraction of modafinil with the one or more pharmaceutically inert carriers and mixing repeatedly until a uniform mixture is formed.

The above co-grinded and/or co-sifted mixture of modafinil and surface active agent and/or pharmaceutical carrier may be further processed with pharmaceutically inert excipients into various dosage forms, such as tablet, capsule, pill and the like, using processes known in the art, for example, by comminuting, mixing, granulating, melting, sizing, filling, drying, molding, immersing, coating, compressing, etc.

In one of the embodiments, the bioavailable dosage form of modafinil may be prepared by a process that includes the steps of blending the above co-grinded and/or co-sifted mixture with one or more extragranular pharmaceutically inert excipients; wet granulating the blend with a granulating fluid or solution/dispersion of one or more pharmaceutically inert excipients in the granulating fluid; drying and sizing the granules; optionally blending with one or more pharmaceutically inert extragranular excipients; and compressing into tablets or filling into capsules.

In another embodiment, the bioavailable dosage form of modafinil may be prepared by a process that includes the steps of blending the above co-grinded and/or co-sifted mixture with one or more extragranular pharmaceutically inert excipients; dry granulating the blend by roller compactor or slugging; sizing the granules; optionally blending with one or more pharmaceutically inert extragranular excipients; and compressing into tablets or filling into capsules.

In yet another embodiment, the bioavailable dosage form of modafinil may be prepared by a process that includes the steps of blending the above co-grinded and/or co-sifted mixture with one or more pharmaceutically inert excipients; and compressing into tablets or filling into capsules.

Dosage forms prepared by any of the above methods may optionally be coated with one or more functional and/or non-functional coatings as desired.

The term “pharmaceutically inert excipients” as used herein includes excipients used in the art of manufacturing solid dosage forms. Examples of pharmaceutically inert excipients include binders, diluents, disintegrants, surface-active agents, lubricants/glidants, coloring agents, and the like.

Examples of suitable binders include methyl cellulose, hydroxypropyl cellulose, hydroxypropyl methylcellulose, povidone, gelatin, gum Arabic, ethyl cellulose, polyvinyl alcohol, pollutant, pregelatinized starch, agar, tragacanth, sodium alginate, propylene glycol, and the like.

Examples of suitable diluents include calcium carbonate, calcium phosphate-dibasic, calcium phosphate-tribasic, calcium sulfate, microcrystalline cellulose, powdered cellulose, dextrates, dextrins, dextrose excipients, fructose, kaolin, lactitol, lactose, mannitol, sorbitol, starch, starch pregelatinized, sucrose, sugar compressible, sugar confectioners, and the like.

Examples of suitable disintegrants include croscarmellose sodium, crospovidone and sodium starch glycolate and the like.

Examples of suitable lubricants/glidants include colloidal silicon dioxide, stearic acid, magnesium stearate, magnesium silicate, hydrogenated vegetable oils, sodium stearyl fumarate, calcium stearate, talc, hydrogenated castor oil, sucrose esters of fatty acid, microcrystalline wax, yellow beeswax, white beeswax, and the like.

Coloring agents include any FDA approved colors for oral use.

Examples of suitable granulating fluids employed in the preparation of dosage forms include methylene chloride, isopropyl alcohol, acetone, methanol, ethanol, water, and the like.

The inventions will be understood more clearly from the following description of the Preparative Examples (Table 1), the dissolution profile (Table 2), and the bioavailability data (Table 3). These examples further exemplify the inventions and are not intended to limit the scope of the inventions.

TABLE 1
Composition Details for Modafinil Tablets
Ingredients (mg/tablet)

| | Ex. 1 | Ex. 2 | Ex. 3 | Ex. 4 | Ex. 5 | Ex. 6 |
|---|---|---|---|---|---|---|
| Intragranular | | | | | | |
| Modafinil (Coarse) | 30 | 30 | 20 | 30 | 30 | 30 |
| Modafinil (Fine) | 170 | 170 | 180 | 170 | 170 | 170 |
| | (d₉₀41, | (d₉₀23, | (d₉₀23, | (d₉₀41, | (d₉₀41, | (d₉₀23, |
| | d₅₀20)* | d₅₀12) | d₅₀12) | d₅₀20) | d₅₀20) | d₅₀12) |
| | **1.3344 | 2.1759 | 2.1759 | | | |
| Colloidal silicon | 10 | 20 | 20 | 10 | 20 | 20 |
| dioxide | | | | | | |
| (Pharmaceutically | | | | | | |
| inert carrier) | | | | | | |
| Sodium Lauryl | — | — | — | 4 | — | 4 |
| sulphate (surface | | | | | | |
| active agent) | | | | | | |
| Polysorbate 80 | — | — | — | — | 5 | — |
| (surface active agent) | | | | | | |
| Lactose | 122 | 112 | 112 | 122 | 112 | 112 |
| Starch | 125 | 125 | 125 | 125 | 125 | 125 |
| Croscarmellose | 10 | 10 | 10 | 10 | 10 | 10 |
| Sodium | | | | | | |
| Povidone | 10 | 10 | 10 | 10 | 10 | 10 |
| Purified water | q.s | q.s | q.s | q.s | q.s | q.s |
| Extragranular | | | | | | |
| Croscarmellose | 10 | 10 | 10 | 10 | 10 | 10 |
| Sodium | | | | | | |
| Colloidal silicon | 5 | 5 | 5 | 5 | 5 | 5 |
| dioxide | | | | | | |
| Talc | 5 | 5 | 5 | 5 | 5 | 5 |
| Magnesium stearate | 2.5 | 2.5 | 2.5 | 2.5 | 2.5 | 2.5 |

*dₓy represents x % of particles with diameter less than or equal to y μm.
**Specific surface area of fine modafinil particles in m²/gm.

### Procedure:

Modafinil tablets according to the above compositions of Examples 1-6 were prepared according to the following method:
a. One or more of mixing and/or co-grinding and/or co-sifting the modafinil and the one or more surface active agents and/or pharmaceutical carriers; granulating with an aqueous solution of binder and optionally surface active agent (Only the fine modafinil particles are mixed with the carrier alone and/or surface active agent);
b. drying the granules;
c. sifting the granules;
d. blending with extragranular inert excipients, and
e. compressing into tablets.

The in vitro release of modafinil from the tablets of Examples 1-6 was studied in USP dissolution apparatus II, in 900 ml water, and at a paddle speed of 25 rpm. The results are presented in Table 2.

TABLE 2
In Vitro Release of Modafinil Tablets

| | Cumulative (%) release of modafinil | | | | | |
|---|---|---|---|---|---|---|
| Time (min) | Ex. 1 | Ex. 2 | Ex. 3 | Ex. 4 | Ex. 5 | Ex. 6 |
| 15 min | 34 | 44 | 42 | 36 | 38 | 45 |
| 30 min | 37 | 56 | 57 | 40 | 41 | 56 |
| 45 min | 41 | 59 | 69 | 45 | 45 | 64 |
| 60 min | 42 | 63 | 72 | 47 | 47 | 67 |
| 90 min | 47 | 69 | 75 | 51 | 51 | 71 |

An in vivo bioequivalence study of the modafinil tablets of Examples 2 and 3 was carried out on healthy male volunteers (n=12) with the marketed Provigil® tablets (200 mg) produced by Abbott Laboratories as the reference. The results of this study are presented in Table 3. The objective of this study was to show that formulations of Examples 2 and 3 provide an activity and safety profile that is similar to or better than that obtained with an equivalent product in the market.

TABLE 3
Comparative Pharmacokinetic Parameters for the Modafinil Tablets of Example 2,
Example 3, and Provigil ®

| Log transformed | | | |
|---|---|---|---|
| ratio of least square | | | |
| means (%) | Cₘₐₓ* | AUC₀₋ₜ** | AUC_{0-∝}*** |
| Modafinil tablet of | 98.52 (91.76-105.79) | 107.51 (94.26-122.62) | 109.34 (94.81-126.09) |
| Example 2/Provigil ® | | | |
| Modafinil tablet of | 94.13 (87.67-101.07) | 110.47 (96.86-126.0) | 107.84 (93.52-124.37) |
| Example 3/Provigil ® | | | |

*Cₘₐₓ = Maximum plasma concentration
**AUC₀₋ₜ = Area under the plasma concentration versus time curve from 0 hours to the time of last sample collected
***AUC_{0-∝} = Area under the plasma concentration versus time curve from 0 hours to infinity
The values in the parenthesis represent the acceptable range of bioequivalence

The results show that modafinil tablets prepared according to Examples 2 and 3 described herein have bioavailability comparable to the reference product, Provigil®.

While several particular forms of the inventions have been described, it will be apparent that various modifications and combinations of the inventions detailed in the text can be made without departing from the spirit and scope of the inventions. For example, the bioavailable dosage forms of modafinil that include modafinil and one or more of a surface active agent and a pharmaceutically inert carrier can be used to treat narcolepsy and/or idiopathic hypersomnia. Moreover, the oral dosage forms of modafinil described herein can be provided with labeling for one or more of wakefulness promotion, to improve wakefulness in patients with excessive daytime sleepiness associated with narcolepsy, and idiopathic hypersomnia. Further, although the examples above are directed to application of the inventive concepts described herein to one particular poorly soluble active pharmaceutical ingredient, these concepts can be applied to other active pharmaceutical ingredients, such antidiabetics, antineoplastic agents, antihypertensives, psychopharmacological agents, cardiovascular agents, platelet aggregation inhibitors, analgesics, antimicrobials, diuretics, spasmolytics, and the like. Specific examples of poorly soluble active pharmaceutical ingredients include glipizide, doxazosin, verapamil, prazosin, isradipine, cilostazol, nifedipine, nisoldipine, bendroflumethazide, chlorpropamide, hydrocortisone, ibuprofen, diclofenac, and the like. Finally, it is contemplated that any single feature or any combination of optional features of the inventive variations described herein may be specifically excluded from the claimed inventions and be so described as a negative limitation. Accordingly, it is not intended that the inventions be limited, except as by the appended claims.

We Claim:

1. An oral dosage form of modafinil comprising modafinil and one or more surface active agents.

2. The oral dosage form of modafinil of claim 1, wherein:
the modafinil comprises fine and coarse modafinil particles;
at least 10% of the modafinil particles comprise coarse modafinil particles and have diameters greater than 220 μm; and
up to 90% of the modafinil particles comprise fine modafinil particles and have diameters less than 220 μm.

3. The oral dosage form of modafinil of claim 1, wherein:
the modafinil comprises fine and coarse modafinil particles;
at least 15% of the modafinil particles comprise coarse modafinil particles and have diameters greater than 220 μm; and
up to 85% of modafinil particles comprise fine modafinil particles and have diameters less than 220 μm.

4. The oral dosage form of modafinil of claim 1, wherein:
the modafinil comprises fine and coarse modafinil particles;
at least 25% of the modafinil particles comprise coarse modafinil particles and have diameters greater than 220 μm; and
up to 75% of the modafinil particles comprise fine modafinil particles and have diameter less than 220 μm.

5. The oral dosage form of modafinil of claim 2, wherein the total specific surface area of the fine modafinil particles is at least 0.2 m²/g.

6. The oral dosage form of modafinil of claim 1, wherein the modafinil and the one or more surface active agents are co-grinded and/or co-sifted.

7. The oral dosage form of modafinil of claim 1, wherein the surface active agent comprises one or more of an anionic, cationic or non-ionic surface active agent.

8. The oral dosage form of modafinil of claim 7, wherein the anionic surface active agent comprises one or more of sodium lauryl sulphate, sodium laurate, dialkyl sodium sulfosuccinates, sodium stearate, potassium stearate, and sodium oleate.

9. The oral dosage form of modafinil of claim 8, wherein the anionic surface active agent comprises sodium lauryl sulphate.

10. The oral dosage form of modafinil of claim 7, wherein the cationic surface active agent comprise one or both of benzalkonium chloride and bis-2-hydroxyethyl oleyl amine.

11. The oral dosage form of modafinil of claim 7, wherein the non-ionic surface active agent comprises one or more of polyoxyethylene sorbitan fatty acid esters, fatty alcohols, glyceryl esters, fatty acid esters of fatty alcohols, and alcohols.

12. The oral dosage form of modafinil of claim 11, wherein the fatty alcohol comprises one or more of lauryl, cetyl and stearyl alcohol.

13. The oral dosage form of modafinil of claim 11, wherein the glyceryl esters comprises one or more naturally occurring monoglycerides, diglycerides and triglycerides.

14. The oral dosage form of modafinil of claim 11, wherein the alcohol is selected from one or more of propylene glycol, polyethylene glycol, sorbitan, sucrose and cholesterol.

15. The oral dosage form of modafinil of claim 11, wherein the polyethylene sorbitan fatty acid ester comprises polysorbate.

16. The oral dosage form of modafinil of claim 1, wherein the amount of surface active agent comprises from about 0.2% to 10% by weight, of the total weight of the dosage form.

17. The oral dosage form of modafinil of claim 1, further comprising one or more pharmaceutically inert carriers, wherein the one or more pharmaceutically inert carriers comprise one or more of cellulose derivatives, silicate derivatives, and clays.

18. The oral dosage form of modafinil of claim 17, wherein the cellulose derivative comprises one or both of microcrystalline cellulose and carboxymethylcellulose.

19. The oral dosage form of modafinil of claim 17, wherein the silicate derivative comprises one or more of magnesium silicate, colloidal silicon dioxide, magnesium trisilicate, and magnesium aluminum silicate.

20. The oral dosage form of modafinil of claim 17, wherein the clay comprises one or more of veegum and bentonite.

21. The oral dosage form of modafinil of claim 1, wherein the amount of pharmaceutically inert carrier comprises from about 2% to about 25% by weight, of total weight of the dosage form.

22. The oral dosage form of modafinil of claim 1, wherein the dosage form comprises a tablet, a capsule, or a pill.

23. The oral dosage form of modafinil of claim 22, wherein the dosage form comprises a tablet.

24. The oral dosage form of modafinil of claim 1, wherein the dosage form further comprises one or more pharmaceutically inert excipients.

25. The oral dosage form of modafinil of claim 24, wherein the pharmaceutically inert excipient comprises one or more of diluents, binders, disintegrants, lubricants/glidants and colors.

26. A process for preparing an oral dosage form of modafinil, the process comprising the steps of:
a. mixing modafinil and one or both of one or more surface active agents and one or more pharmaceutically inert carriers;
b. grinding and/or sifting the mix of step a;
c. combining with pharmaceutically inert excipients; and
d. compressing or filling into a suitable dosage form.

27. The process according to claim 26, wherein:
the modafinil comprises fine and coarse modafinil particles;
at least 10% of the modafinil particles comprise coarse modafinil particles and have bn diameters greater than 220 μm; and
up to 90% of the modafinil particles comprise fine modafinil particles and have diameters less than 220 μm.

28. The process according to claim 26, wherein:
the modafinil comprises fine and coarse modafinil particles;
at least 15% of the modafinil particles comprise coarse modafinil particles and have diameters greater than 220 μm; and
up to 85% of the modafinil particles comprise fine modafinil particles and have diameters less than 220 μm.

29. The process according to claim 27, wherein:
the modafinil comprises fine and coarse modafinil particles;
at least 25% of the modafinil particles comprise coarse modafinil particles and have diameters greater than 220 μm; and
up to 75% of the modafinil particles comprise fine modafinil particles and have diameters less than 220 μm.

30. The process according to claim 26, wherein the total specific surface area of the fine modafinil particles is at least 0.2 m²/g.

31. The process according to claim 26, wherein the dosage form comprises one or more of a tablet, a capsule, and a pill.

32. The process according to claim 31, wherein the dosage form comprises a tablet.

33. The process according to claim 32, wherein the tablet is prepared by one or more of a process of wet granulation, dry granulation, or direct compression method.

34. The process according to claim 33, wherein the tablet is prepared by a wet granulation method.

35. The process according to claim 33, wherein the tablet is prepared by a dry granulation method.

36. The process according to claim 33, wherein the tablet is prepared by a direct compression method.

37. The process according to claim 31, wherein the dosage form comprises a capsule.

38. The process according to claim 32, wherein the dosage form is coated with one or more functional and/or non-functional layers.

39. A method of treating one or both of narcolepsy and idiopathic hypersomnia by administering an oral dosage form of modafinil, the dosage form comprising coarse and fine modafinil particles and one or more surface active agents, wherein the fine modafinil particles have diameters less than 220 μm.

40. The method according to claim 39, wherein at least 10% of the modafinil particles have diameters greater than 220 μm.

41. The method according to claim 40, wherein at least 15% of the modafinil particles have diameters greater than 220 μm.

42. The method according to claim 41, wherein at least 25% of the modafinil particles have diameters greater than 220 μm.

43. The method according to claim 39, wherein the total specific surface area of the fine modafinil particles is at least 0.2 m²/g.

44. A mixture comprising modafinil particles and one or both of one or more surface active agents and one or more pharmaceutically inert carriers, wherein the mixture is one or both of co-grinded and co-sifted.

45. The mixture according to claim 44, wherein:
at least 10% of the modafinil particles are coarse and have diameters greater than 220 μm; and
up to 90% of the modafinil particles are fine and have diameters less than 220 μm.

46. The mixture according to claim 45, wherein:
at least 15% of the modafinil particles are coarse and have diameter greater than 220 μm; and
up to 85% of the modafinil particles are fine having diameter less than 220 μm.

47. The mixture according to claim 46, wherein:
at least 25% of the modafinil particles are coarse and have diameters greater than 220 μm; and
up to 75% of the modafinil particles are fine and have diameters less than 220 μm.

48. The mixture according to claim 44, wherein the total specific surface area of the fine modafinil particles is at least 0.2 m/g, the fine modafinil particles having diameters less than 220 μM.

49. An oral dosage form of modafinil comprising modafinil and one or more surface active agents, wherein the one or more surface active agents comprises one or more of an anionic, cationic or non-ionic surface active agent.

50. The oral dosage form of modafinil of claim 49, wherein:
the modafinil comprises fine and coarse modafinil particles;
at least 10% of the modafinil particles comprise coarse modafinil particles and have diameters greater than 220 μm; and
up to 90% of the modafinil particles comprise coarse modafinil particles and have diameters less than 220 μm.

51. The oral dosage form of modafinil of claim 49, wherein: p1 the anionic surface active agent comprises one or more of sodium lauryl sulphate, sodium laurate, dialkyl sodium sulfosuccinates, sodium stearate, potassium stearate, and sodium oleate;
the cationic surface active agent comprises one or both of benzalkonium chloride and bis-2-hydroxyethyl oleyl amine; and
the non-ionic surface active agent comprises one or more of polyoxyethylene sorbitan fatty acid esters, fatty alcohols, glyceryl esters, fatty acid esters of fatty alcohols, and alcohols.

52. The oral dosage form of modafinil of claim 50, further comprising one or more pharmaceutically inert carriers, wherein the one or more pharmaceutically inert carriers comprise one or more of cellulose derivatives, silicate derivatives, and clays.

53. The oral dosage form of modafinil of claim 49, further comprising one or more additional active pharmaceutical ingredients.

54. An oral dosage form of modafinil comprising modafinil and one or both of one or more surface active agents and one or more pharmaceutically inert carriers;
wherein the one or more surface active agents comprise one or more of an anionic, cationic or non-ionic surface active agent; and
wherein the one or more pharmaceutically inert carrier comprise clay.
